(19) United States
(12) Patent Application Publication
(10) Pub. No.: US 2004/0097979 A1
(43) Pub. Date: May 20, 2004

(54) Aortic valve implantation device

(76) Inventors: Oleg Svanidze, Irvine, CA (US); Rodolfo C. Quijano, Laguna Hills, CA (US); Hosheng Tu, Newport Beach, CA (US)

Correspondence Address: JONES DAY, 555 WEST FIFTH STREET, SUITE 4600, LOS ANGELES, CA, 90013-1025, US

(21) Appl. No.: 10/294,772

(22) Filed: Nov. 14, 2002

(51) Int. Cl. A61B 17/08

(52) U.S. Cl. 606/151

(57) ABSTRACT

This invention discloses a miniclip apparatus for releasably stabilizing a leaflet onto an aortic artery wall during aortic valve implantation comprising a clip base having a first clip member consisting of a plurality of first prongs and an opposite second clip member consisting of a plurality of second prongs, wherein the first prongs and the second prongs are sized and configured for releasably clipping and stabilizing the leaflet in conjunction with the aortic artery wall; and an actuator assembly operable using one hand located at the clip base, wherein the first clip member moves away from the second clip member when the actuator assembly is activated.

### FIELD OF THE INVENTION

The present invention relates to fastening devices and a method for assisting implantation of an aortic bioprosthetic valve in a body channel, and more particularly, to reusable miniclip apparatuses to facilitate orienting and releasably securing bioprosthetic heart valve leaflets during the valve implantation.

### BACKGROUND OF THE INVENTION

Various surgical techniques may be used to repair a diseased or damaged valve, including annuloplasty (contracting the valve annulus), quadrangular resection (narrowing the valve leaflets), commissurotomy (cutting the valve commissures to separate the valve leaflets), or decalcification of valve and annulus tissue. Alternatively, the valve may be replaced, by excising the valve leaflets of the natural valve, and securing a replacement valve in the valve position, usually by suturing the replacement valve to the natural valve annulus.

Prosthetic heart valves are used to replace damaged or diseased human heart valves. The heart is a hollow muscular organ having four pumping chambers: the left and right atria and the left and right ventricles, each provided with its own one-way valve. Human heart valves under the conditions of normal physiological functions are passive devices that open under the pressure of blood flow on their leaflets. There are four valves in the heart that serves to direct the flow of blood through all chambers in a forward direction.

In general, blood leaves the heart lower chambers in the direction to the rest of the body or to the lungs for required oxygenation, or blood enters the lower chambers from the upper chambers of the heart. Similarly, they close under the pressure exerted on the same leaflet elements when blood flow is retrograde, thus impeding return of blood flow to the chamber it has just left. This, under normal conditions, (that is, when the body is not under physical stresses and the heart is beating at the normal resting state of about 70 beats per minute) equates to the leaflets opening by separation from each other, thereby producing an opening or closing by apposing to each other approximately 38 million times per year. It can be surmised that under stress conditions this may be happening at higher rates, thus increasing the number of separations and appositions, as well as the forces of impact between the leaflets during the closing. Prosthetic heart valves can be used to replace any of these naturally occurring valves, although repair or replacement of the aortic or mitral valves is most common because they reside in the left side of the heart where pressures are the greatest.

When disease conditions affect the structure of the materials of the components of the native valve apparatus, the valve itself will decay, degenerate or disrupt and require repair or replacement to restore proper function necessary for the continuation of life.

Where replacement of a heart valve is indicated, the dysfunctional valve is typically cut out and replaced with either a mechanical valve, or a tissue valve. Tissue valves are often preferred over mechanical valves because they typically do not require long-term treatment with anticoagulants. The most common tissue valves are constructed with whole porcine (pig) valves, or with separate leaflets cut from bovine (cow) or equine (horse) pericardium. U.S. Pat. No. 6,461,382, entire contents of which are incorporated herein by reference, discloses a typical flexible heart valve construct with reduced vibration-related strain.

Cox in U.S. Pat. No. 6,270,526, entire contents of which are incorporated herein by reference, discloses a replacement aortic valve with the inlet end of a tubular segment sutured to the valve annulus while the outlet end of the tube is sutured longitudinally along three lines. It is one aspect of the present invention to simplify the suturing operation of the outlet end via reusable miniclip apparatuses to facilitate accurately and precisely orienting and releasably securing bioprosthetic heart valve leaflets during the valve implantation.

The open-heart valve replacement is a long tedious procedure. For implantation of a bioprosthetic valve in the aortic position, a surgeon typically opens the aorta and excises the native valve. The surgeon then inserts the prosthetic valve through the opening in the aortic wall and secures the prosthesis at the junction of the aorta and the left ventricle. The inflow annulus of the valve faces the left ventricle and, relative to the surgeon's perspective, may be termed the distal annulus, while the outflow annulus of the valve faces the aorta and may be termed the proximal annulus.

Cosgrove et al. in U.S. Pat. No. 6,197,053, entire contents of which are incorporated herein by reference, discloses a holding apparatus for facilitating implantation of a prosthetic heart valve within a heart, the apparatus comprising a cage having a prosthesis retention space and is releasably attached to the proximal end of the heart valve prosthesis. The releasable attachment of the prosthesis to the holding apparatus may be accomplished by a number of suture threads which are passed through the prosthesis and threaded upon the holding apparatus. Such a holding apparatus is bulky and difficult to operate within a confined heart valve space.

After the prosthetic tissue valve ring is placed and implanted in the aortic annulus position, the leaflets need to be attached to the aorta. A conventional procedure for releasably securing the commissure of the leaflets to the artery wall is usually accomplished by a clamp followed by suturing. Since the commissures are oriented toward the artery wall one at a time, the relative location of the commissures onto the aortic artery temporarily held by an atraumatic clamp may be re-positioned several times for intended spacing apart and fastening, which exposes the patient to unnecessary longer surgery duration. Therefore, it would be desirable to provide a reusable miniclip apparatus that is simple, useful, less expensive to manufacture, and easy to use so as to overcome the disadvantages of the current clamping practice. The improved miniclip apparatus is to facilitate precisely and accurately orienting and releasably securing a bioprosthetic heart valve leaflet during the valve implantation that saves time of the open-chest operation.

### SUMMARY OF THE INVENTION

It is one object of the present invention to provide a miniclip apparatus for releasably stabilizing a leaflet onto an aortic wall during an aortic valve implantation. In one aspect, the miniclip apparatus comprises a clip base having a first clip member consisting of a plurality of first prongs and an opposite second clip member consisting of a plurality of second prongs, wherein the first prongs and the second prongs are sized and configured for releasably clipping and stabilizing the leaflet in conjunction with the aortic wall. In one embodiment, the first clip member is configured essentially parallel to the second clip member. The aortic valve herein may be a porcine valve or a valve fabricated from pericardium tissue selected from a group consisting of equine, bovine, porcine, and ovine.

In another aspect, the miniclip apparatus further comprises an actuator assembly operable using one hand, the actuator assembly being located at the clip base, wherein the first clip member moves away from the second clip member when the actuator assembly is activated. In one embodiment, the first clip member and the second clip member are preshaped and configured enabling the two clip members to clip and stabilize the leaflet in conjunction with the aortic artery wall when the actuator assembly is not activated. In a particular embodiment, the actuator assembly is absent of a coiled spring construct.

The plurality of first prongs of the miniclip apparatus further comprises a first set of prongs and a second set of prongs, and wherein a proper distance is configured between the first set and the second set of prongs for releasably holding a pledget therebetween, and wherein the proper distance is increased when the actuator assembly is activated.

It is another object of the present invention to provide a method for releasably stabilizing three leaflets of an aortic valve onto an aortic artery wall during aortic valve implantation. The method comprises orienting all three commissures of the three leaflets toward the aortic artery wall to form double-layer composites spaced apart at about 120 degrees, each double-layer composite having an interior side and an exterior side. In one aspect, the method further comprises selecting miniclip apparatus and activating the actuator assembly of the miniclip apparatus while simultaneously inserting the miniclip apparatus over the double-layer composite, wherein the first clip member lies on the interior side of the composite and the second clip member lies on the exterior side of the composite. Finally, the method comprises a step of passing a suture through the three-layer composite and deactivating the actuator assembly to releasably clipping and stabilizing the first leaflet in conjunction with the aortic artery wall.

### BRIEF DESCRIPTION OF THE DRAWINGS

Additional objects and features of the present invention will become more apparent and the invention itself will be best understood from the following Detailed Description of Exemplary Embodiments, when read with reference to the accompanying drawings.

FIG. 1 is a reusable miniclip apparatus to facilitate locating, orienting and releasably securing a bioprosthetic heart valve leaflet during the valve implantation in accordance with one embodiment of the present invention.

FIG. 2 is a simple miniclip apparatus of FIG. 1 at a released state.

FIG. 3 is a prior art clipping using a clamp for holding the valve leaflet and a portion of the aortic artery wall together during implantation of an aortic valve in a body channel.

FIG. 4 is an illustrative example of the current device holding a pledget as part of the aortic valve leaflet fastening procedures.

FIG. 5 is another illustration of applying the miniclip apparatus for holding the valve leaflet and a portion of the aortic artery wall together for fastening.

FIG. 6 is a traverse cross-sectional view of the composite to be sutured together, section 1-1 of FIG. 5.

### DETAILED DESCRIPTION OF EXEMPLARY EMBODIMENTS

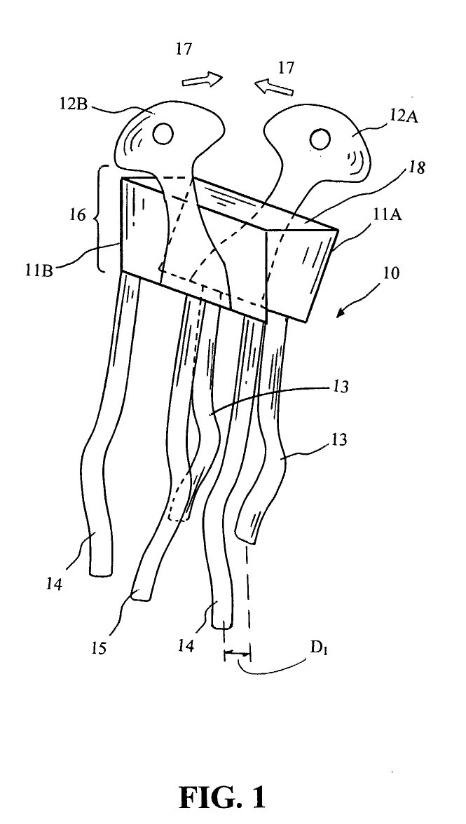

Referring to FIGS. 1 to 6, what is shown is an embodiment of a releasably fastening device used in aortic valve implantation, wherein the device is to facilitate accurate and quick locating, orienting, and releasably securing bioprosthetic heart valve leaflets during the valve implantation. While the description sets forth various embodiment specific details, it will be appreciated that the description is illustrative only and should not to be construed in any way as limiting the invention. Furthermore, various applications of the invention, and modifications thereto, which may occur to those who are skilled in the art, are also encompassed by the general concepts described below.

Aortic stenosis is a disease of the aortic valve in the left ventricle of the heart. This aortic valvular orifice can become tightly stenosed, and therefore the blood cannot anymore be freely ejected from the left ventricle. In fact, only a reduced amount of blood can be ejected by the left ventricle which has to markedly increase the ventricular chamber pressure to pass the stenosed aortic orifice. In such aortic diseases, the patients can have syncope, chest pain, and mainly difficulty in breathing. Aortic stenosis is a very common disease in people above sixty years old and occurs more and more frequently as the subject gets older. The evolution of such a disease is disastrous when symptoms of cardiac failure appear and many patients die in the year following the first symptoms of the disease. The commonly available treatment is the replacement of the stenosed aortic valve by a prosthetic valve via open-heart surgery.

The natural leaflets include arcuate cusp portions separated by common commissure portions. If the natural valve has three leaflets, and has a vertically oriented flow axis, the leaflets are evenly distributed circumferentially 120 degrees apart with lower cusp portions and upstanding commissure portions. The commissure portions are connected between the cusp portions and are generally axially aligned along the aortic wall. The annular root of an aortic valve is composed of fibrous tissue and generally conforms to the undulating perimeter of the valve to support the leaflets.

Carpentier in U.S. Pat. No. 6,338,740, entire contents of which are incorporated herein by reference, discloses a heart valve with radially moveable cusps and commissures wherein the commissures may be pivotally or flexibly coupled. Carpentier '740 also discloses a multi-legged holder having legs alternating between each cusp and commissure to be used in the implantation. Brendzel et al. in U.S. Pat. No. 6,391,053, entire contents of which are incorporated herein by reference, discloses a prosthetic heart valve having valve housing and a cuff positioned such that prosthesis is attached in a supraannular position relative to a tissue annulus of the heart. Neither patent discloses a simple miniclip apparatuses to facilitate orienting and releasably securing bioprosthetic heart valve leaflets during the valve implantation.

The tissue valve or tissue valve leaflets are generally chemically treated to render the valve suitable for long-term implantation in human. Glutaraldehyde is a chemical most often used for tissue fixation. The tissue fixation is well known to an ordinary artisan who is skilled in the art and does not constitute a part of the present invention.

In this respect, implanting the aortic heart valve of the present invention involves excising the natural leaflets and attaching the prosthetic heart valve proximate the fibrous annulus, but also in part up the aortic wall. The attachment means may be sutures, staples, adhesives, or otherwise, that is anchored into the aortic wall itself, adjacent to the fibrous annulus.

Suture is biocompatible, flexible and long lasting. The suture arrangement useful in the present invention comprises a first needle and a second needle connected by length of suture. After passing the first and the second needles from within the aorta through the wall of aorta and valve leaflet outwardly, the needles may then be pulled away from the aorta wall to thread the suture through the tissue.

FIG. 1 shows a simple miniclip apparatus to facilitate accurately and quickly orienting and releasably securing a bioprosthetic heart valve leaflet during the valve implantation in accordance with one embodiment of the present invention. The miniclip apparatus is absent of a coiled spring or other complicate structure that may retain debris from previous surgeries, even after autoclaving.

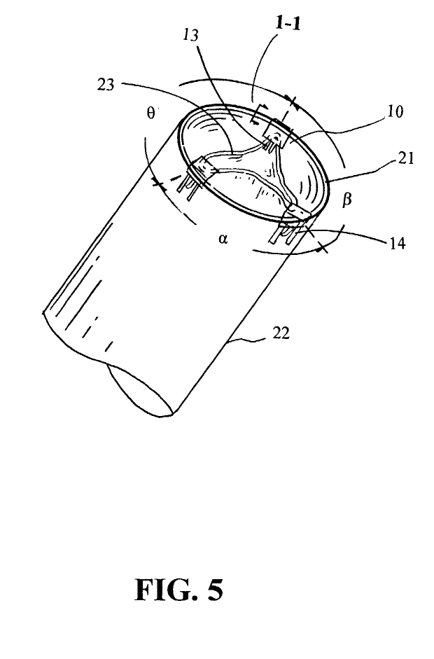

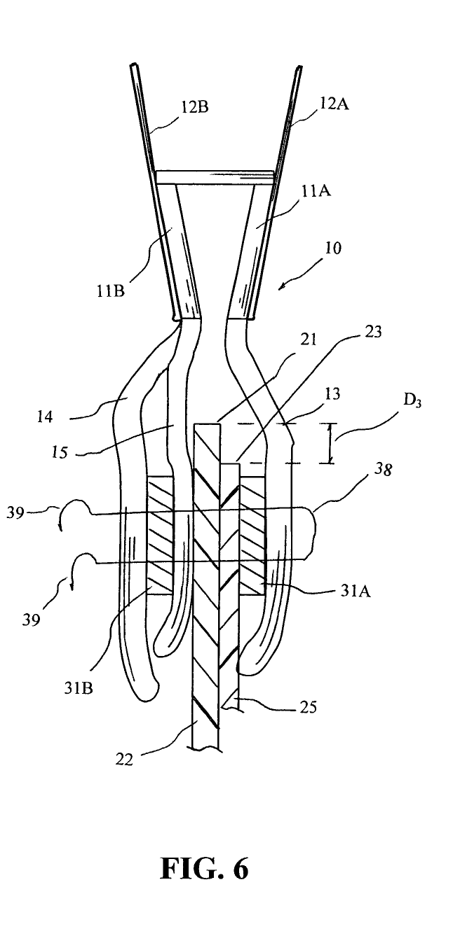

The miniclip apparatus 10 of the present invention for releasably stabilizing or fixing a leaflet onto an aortic artery wall during aortic valve implantation may comprise a clip base 16 having a first clip member 11B consisting of a plurality of first prongs (14 and 15) and an opposite second clip member 11A consisting of a plurality of second prongs 13, wherein the first prongs (14, 15) and the second prongs 13 sized and configured for releasably clipping and stabilizing the leaflet 25 in conjunction with the aortic artery wall 22 (shown in FIGS. 5 and 6). The miniclip apparatus 10 further comprises an actuator assembly 12A, 12B operable using one hand located at the clip base 16, wherein the first clip member 11B moves away from the second clip member 11A when the actuator assembly 12A/12B is activated. The first clip member 11B and the second clip member 11A are connected through a middle member 18 with a preset spring effect. One method for activating the actuator assembly is to press the assembly elements 12A and 12B toward each other as shown by an arrow 17 in FIG. 1.

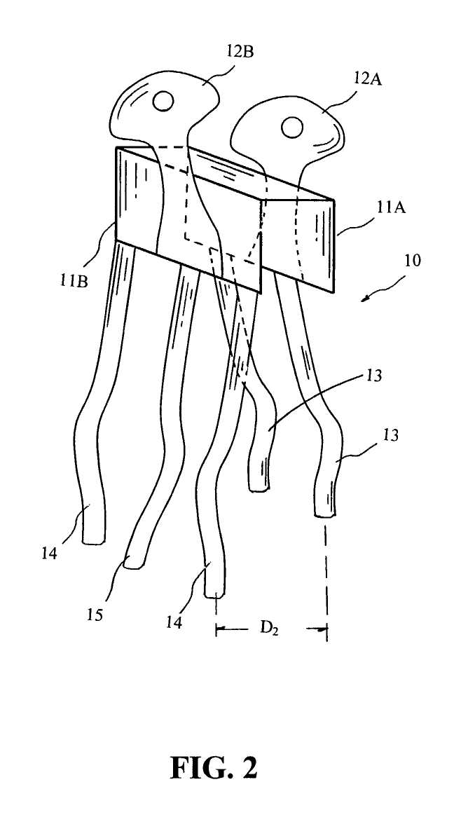

In a further aspect of the present invention, the first clip member is configured essentially parallel to the second clip member. In another aspect, the first clip member and the second clip member are preshaped and configured enabling the two clip members to clip and stabilize the leaflet in conjunction with the aortic artery wall when the actuator assembly is not activated. Elements of the miniclip may be made of stainless steel, Nitinol or other suitable metal that could be preshaped and configured with the intended clipping properties. In some aspect, the plurality of first prongs further comprises a first set of prongs 14 and a second set of prongs 15, and wherein a proper distance, D₁, is sized and configured between the first set 14 and the second set 15 of prongs for releasably holding a pledget 31 therebetween. The proper distance D₁ is sized and configured to snugly hold the pledget 31. The proper distance is increased from D₁ of FIG. 1 to D₂ of FIG. 2 when the actuator assembly is activated. FIG. 2 shows a simple miniclip apparatus of FIG. 1 at a released state when the actuator assembly is activated.

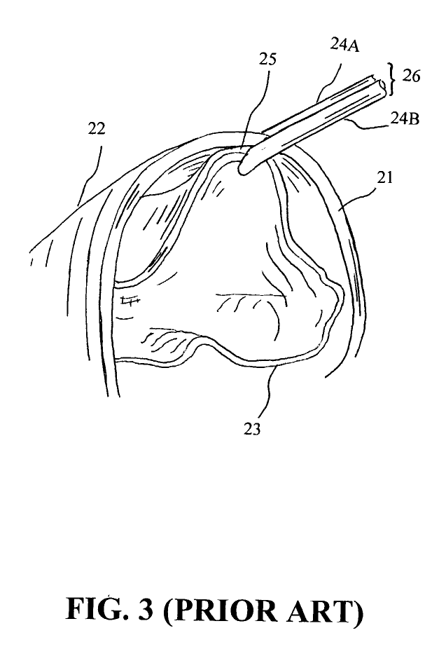

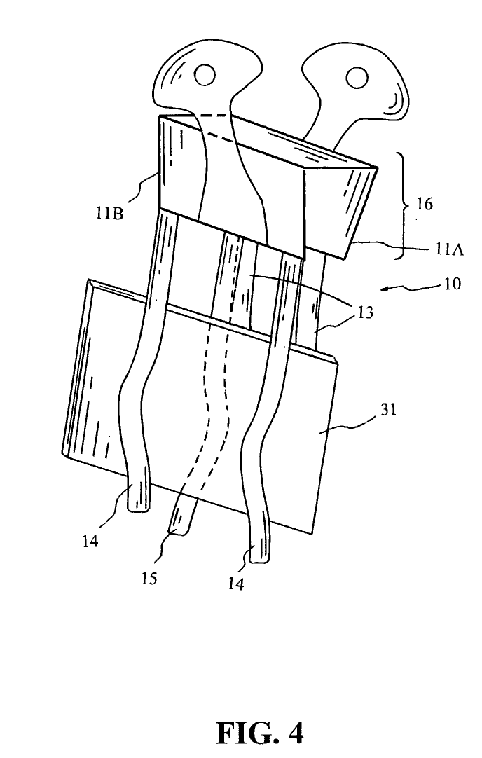

FIG. 3 is a prior art clipping illustration using a clamp 26 for holding the valve leaflet 25 and a portion of the aortic artery wall 22 together during implantation of an aortic valve in a body channel. The clamp 26 generally includes two jaws 24A, 24B that may have a wide variety of preset clamping pressures, which are mostly used for vessel occlusion. During operations, one hand is needed to hold the clamp 26 for fastening purposes. The conventional clamp does not have additional features of holding at least one pledget along with the general releasably clipping function as shown in FIG. 4, wherein the miniclip of the present invention is simply lightweight and can be left alone without a hand to hold it.

FIG. 4 is an illustrative example of the current device 10 holding a pledget 31 as part of the aortic valve leaflet fastening procedures. FIG. 5 shows another illustration of applying the miniclip apparatus 10 for holding the valve leaflet 25 and a portion of the aortic artery wall 22 together for fastening. In operations, the miniclips each holding the composite of a commissure of one leaflet toward the aortic artery wall can be placed at the edge 21 of the aortic artery wall 22 at an angle α, β, and θ, wherein each angle of α, β, or θ may be about 120 degrees or with any predetermined angles.

FIG. 6 shows a traverse cross-sectional view of the composite to be sutured together, section 1-1 of FIG. 5. The composite comprises a first set of prongs 14 and a second set of prongs 15 sandwiching a first pledget 31B. The composite further comprises the combined set of prongs 14/15 and the plurality 13 of second prongs sandwiching the aortic artery wall 22, the commissure portion of the leaflet 25 and optionally a second pledget 31A. In operations, the composite is temporarily held by a miniclip 10 of the present invention and is ready for passing a suture to fasten the composite together. After fastening, the miniclip 10 is easily released from the composite by slightly activating the actuator assembly 12A/12B. In another aspect, the miniclip is to releasably stabilize and hold the composite that comprises a synthetic tab that is securely attached to the distal end of the leaflet, rather than the leaflet itself, wherein the synthetic tab may be made of expanded polytetrafluoroethylene (Teflon™), polyester (Dacron™), silicone (Silastic™), polyurethane (Pellethane™) or other suitable synthetic material.

The edge 23 of the commissure 25 is generally oriented at a distance D₃ lower than the edge 21 of the aortic artery wall 22. The distance D₃ is at least one millimeter, preferably at 2-3 millimeters.

It is one aspect of the present invention to utilize the miniclip 10 of the present invention for assisting the aortic valve implantation. Therefore, it is one object of the present invention to provide a method for releasably stabilizing three leaflets of an aortic valve onto an aortic artery wall during aortic valve implantation comprising: (a) orienting a commissure of one of the three leaflets toward the aortic artery wall to form a double-layer composite, having an interior side and an exterior side; (b) selecting one miniclip apparatus of claim 1; (c) activating the actuator assembly of the miniclip apparatus while simultaneously inserting the miniclip apparatus over the double-layer composite, wherein the first clip member lies on the interior side of the composite and the second clip member lies on the exterior side of the composite; (d) deactivating the actuator assembly to releasably clipping and stabilizing the first leaflet in conjunction with the aortic artery wall; and (e) repeating the steps of (a) to (d) for additional two miniclip apparatuses on the remaining two leaflets, wherein the three miniclip apparatuses are spaced apart at about 120 degrees or any predetermined angle.

In one aspect, the method may further comprise, after the step (a), a step of inserting at least a pledget along with at least one of the double-layer composites to form a three-layer composite or a multiple-layer composite, the multiple-layer composite having an interior side and an exterior side. The pledget may be selected from a group consisting of an expanded polytetrafluoroethylene (Teflon™), polyester (Dacron™), silicone (Silastic™), polyurethane (Pellethane™) or other suitable synthetic material.

In another aspect, the method may further comprise, after the step (e), a step of passing a suture through the three-layer or multiple-layer composite, wherein the step of passing a suture may be carried out by passing a needle of the suture from the anterior side of the multiple-layer composite. The method may further comprise a step of passing a second needle of the suture from the anterior side of the multiple-layer composite, followed by a step of removing the miniclip apparatus from the multiple-layer composites.

In one embodiment, the method may further comprise a step of removing at least a portion of a patient's heart valve by means of a cutting tool. In some aspect of the present invention, the cutting tool may be made of an electrically conductive metal and radiofrequency energy is provided to the cutting tool for enhanced valve removal. The high frequency energy ablation is well known to an ordinary artisan who is skilled in the art.

In operations, the step of orienting the commissure of the leaflets against the aortic artery wall may be carried out by inserting a dilator into a center of the aortic valve. The dilator can be a balloon-based device or a basket-type expandable device. The dilator and its use are well known to an ordinary artisan skilled in the art.

From the foregoing description, it should now be appreciated that a miniclip apparatuses to facilitate locating, orienting, and releasably securing bioprosthetic heart valve leaflets during the valve implantation and method of use thereof have been disclosed. While the invention has been described with reference to a specific embodiment, the description is illustrative of the invention and is not to be construed as limiting the invention. Various modifications and applications may occur to those who are skilled in the art, without departing from the true spirit and scope of the invention, as described by the appended claims.

What is claimed is:

1. A miniclip apparatus for releasably stabilizing a leaflet onto an aortic artery wall during aortic valve implantation comprising:
a clip base having a first clip member consisting of a plurality of first prongs and an opposite second clip member consisting of a plurality of second prongs, wherein the first prongs and the second prongs are sized and configured for releasably clipping and stabilizing the leaflet in conjunction with the aortic artery wall; and
an actuator assembly operable using one hand located at the clip base, wherein the first clip member moves away from the second clip member when the actuator assembly is activated.

2. The miniclip apparatus of claim 1, wherein the first clip member is configured essentially parallel to the second clip member.

3. The miniclip apparatus of claim 1, wherein the first clip member and the second clip member are preshaped and configured enabling the two clip members to clip and stabilize the leaflet in conjunction with the aortic artery wall when the actuator assembly is not activated.

4. The miniclip apparatus of claim 1, wherein the plurality of first prongs further comprises a first set of prongs and a second set of prongs, and wherein a proper distance is configured between the first set and the second set of prongs for releasably holding a pledget therebetween.

5. The miniclip apparatus of claim 3, wherein the proper distance is increased when the actuator assembly is activated.

6. The miniclip apparatus of claim 1, wherein the actuator assembly is absent of a coiled spring.

7. The miniclip apparatus of claim 1, wherein the aortic valve is a tissue valve fabricated from a porcine heart valve.

8. The miniclip apparatus of claim 1, wherein the aortic valve is a tissue valve fabricated from equine pericardia.

9. The miniclip apparatus of claim 1, wherein the aortic valve is a tissue valve fabricated from bovine pericardia.

10. A method for releasably stabilizing three leaflets of an aortic valve onto an aortic artery wall during aortic valve implantation comprising:
(a) orienting a commissure of one of the three leaflets toward the aortic artery wall to form a double-layer composite, having an interior side and an exterior side;
(b) selecting one miniclip apparatus of claim 1;
(c) activating the actuator assembly of the miniclip apparatus while simultaneously inserting the miniclip apparatus over the double-layer composite, wherein the first clip member lies on the interior side of the composite and the second clip member lies on the exterior side of the composite;
(d) deactivating the actuator assembly to releasably clipping and stabilizing the first leaflet in conjunction with the aortic artery wall;
(e) repeating the steps of (a) to (d) for additional two miniclip apparatuses on the remaining two leaflets, wherein the three miniclip apparatuses are spaced apart at about 120 degrees.

11. The method of claim 10, wherein after the step (a) further comprises a step of inserting at least a pledget along with at least one of the double-layer composites to form a three-layer composite, the three-layer composite having an interior side and an exterior side.

12. The method of claim 11, wherein the pledget is an expanded polytetrafluoroethylene.

13. The method of claim 11, wherein after the step (e) further comprises a step of passing a suture through the three-layer composite.

14. The method of claim 13, wherein the step of passing a suture is carried out by passing a needle of the suture from the anterior side of the three-layer composite.

15. The method of claim 14 further comprising a step of passing a second needle of the suture from the anterior side of the three-layer composite.

16. The method of claim 15 further comprising a step of removing the miniclip apparatus from the three-layer composites.

17. The method of claim 10, wherein the step of orienting the commissure of the leaflets against the aortic artery wall is carried out by inserting a dilator into a center of the aortic valve.

18. The method of claim 10, wherein an edge of the commissure is oriented at a distance lower than an edge of the aortic artery wall.

19. The method of claim 18, wherein said distance is at least one millimeter.

20. The method of claim 10, wherein the aortic valve is a tissue heart valve selected from a group consisting of a porcine heart valve, a bovine pericardium valve, and an equine pericardium valve.
